(19) United States
(12) Patent Application Publication
(10) Pub. No.: US 2006/0212065 A1
(43) Pub. Date: Sep. 21, 2006

(54) Anus insert for cryotherapy

(76) Inventors: Shu-Wen Jao, Taipei (TW); Wei Lee, Taipei (TW)

Correspondence Address: TROXELL LAW OFFICE PLLC, SUITE 1404, 5205 LEESBURG PIKE, FALLS CHURCH, VA, 22041, US

(21) Appl. No.: 11/082,823

(22) Filed: Mar. 18, 2005

(51) Int. Cl. A61M 29/00

(52) U.S. Cl. 606/197

(57) ABSTRACT

An anus insert for hemorrhoid treatment is disclosed to have a cylindrical projection for insertion into the anus, a disk-like head disposed at one end of the cylindrical projection for the holding of the user's hand to insert the cylindrical projection into the anus and defining with the cylindrical projection an enclosed space, and a non-toxic low freezing point fluid filled in the enclosed space. The anus insert is kept in a refrigerator to lower its temperature to or below the freezing point before use and washable for repeated use at home.

### BACKGROUND OF THE INVENTION

1. Field of the Invention

The present invention relates generally to an implement for cryotherapy and more particularly, to an anus insert for cryotherapy.

2. Description of the Related Art

Cryotherapy is a method that uses cold temperature to treat hemorrhoids. By means of applying cold or refrigeration to hemorrhoids, cryotherapy relieves pain; and enhances anus tissues to contract hemorrhoids. In hospitals, liquefied nitrogen or CO₂ is used as refrigerate for cryotherapy to freeze hemorrhoids, causing hemorrhoids to decay and to drop. The temperature of liquefied nitrogen or CO₂ can be as low as −30° C.˜−37° C. This low temperature may destruct anus tissues, causing the skin around the anus area to decay and to further release secretion and bad smell for as long as 8 weeks. Therefore, this method is rarely accepted.

There is reported a cryotherapy in which a surgical glove is filled with water and tied up, and then frozen, and then one frozen finger portion of the glove is inserted into the anus to treat hemorrhoids. This method is inconvenient to perform.

In addition to cryotherapy, there are other hemorrhoids treatment methods including rubber band ligature, infrared burning, sclerosing agent injection, and etc. These methods are not within the scope of the discussion to which the present invention pertains. Therefore, no further detailed description in this regard is necessary.

### SUMMARY OF THE INVENTION

According to one aspect of the present invention, the anus insert comprises a cylindrical projection, a disk-like head disposed at one end of the cylindrical projection, and a low freezing point fluid filled in an enclosed space defined inside the disk-like head and the cylindrical projection. The anus insert is kept in a refrigerator to lower its temperature to or below the freezing point before use. When inserted into the patient's anus, the low freezing point fluid starts to defreeze gradually without causing the skin of the anus to rote. Therefore, the anus insert is suitable for a long use at home to relieve pain, and to cause creation of dense, firm scar tissue in response to previous tissue disease. According to another aspect of the present invention, the low freezing point fluid can be glycerin, water, or anti-freezing agent, and the anus insert is washer for use repeatedly. According to still another aspect of the present invention, the anus insert is washable for repeated use. According to still another aspect of the present invention, the disk-like head is maintained in close contact with external hemorrhoids after insertion of the cylindrical projection into the anus. Therefore, the anus insert is suitable for treating external hemorrhoids as well as internal hemorrhoids.

### BRIEF DESCRIPTION OF THE DRAWINGS

FIG. 1 is an elevational view of an anus insert according to the present invention.

FIG. 2 is a sectional view of the anus insert according to the present invention.

FIG. 3 is an exploded view of an alternate form of the anus insert according to the present invention.

FIG. 4 is an assembly view of FIG. 3.

FIG. 5 is a schematic sectional view of the present invention after filling of a low freezing point fluid in the anus insert.

FIG. 6 corresponds to FIG. 5, showing the filling hole sealed with a heat-sealing apparatus.

### DETAILED DESCRIPTION OF THE INVENTION

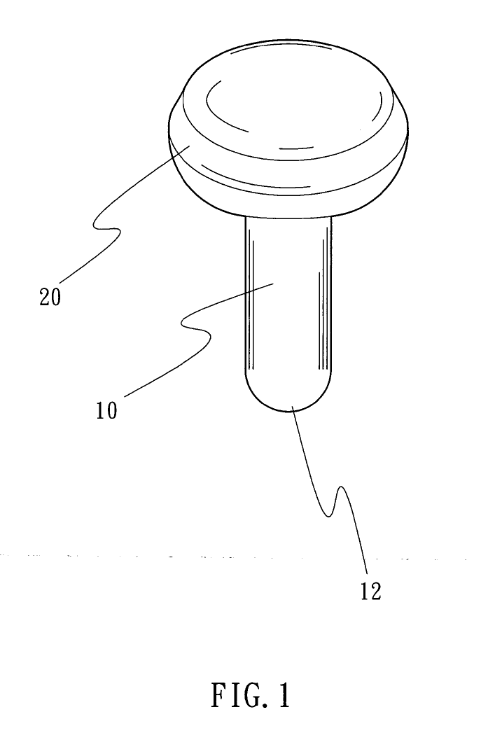

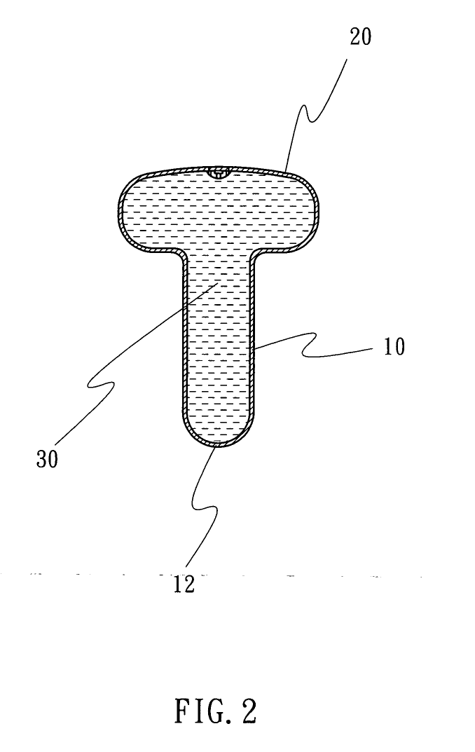

Referring to FIGS. 1 and 2, an anus insert is a hollow member comprising a cylindrical projection 10 and a disk-like head 20 at one end of the cylindrical projection 10. The cylindrical projection 10 has a smooth outer surface and is terminated at an outwardly curved arched portion 12. The hollow anus insert is filled up with a low freezing point fluid 30. After filling of the low freezing point fluid 30 into the hollow anus insert, the anus insert is well sealed.

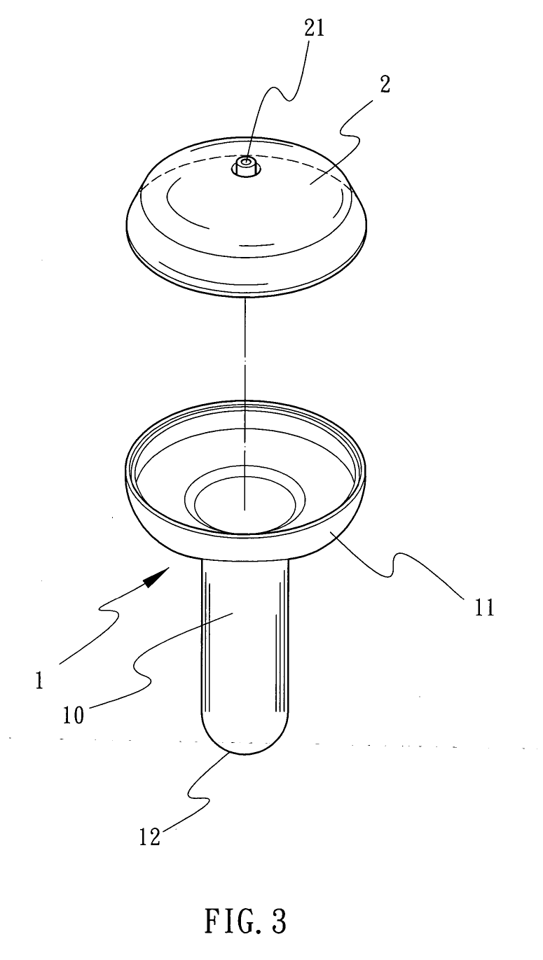

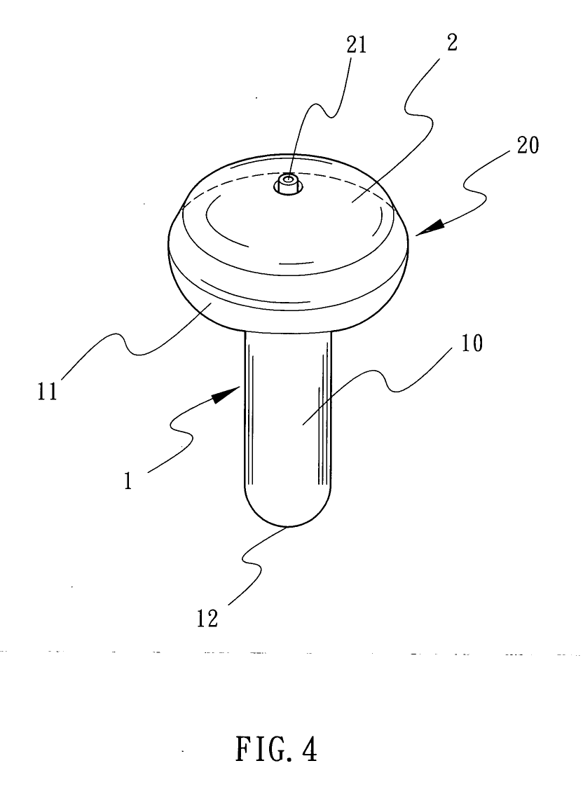

FIGS. 3 and 4 show an alternate form of the present invention. According to this embodiment, the anus insert is comprised of a body member 1 and a cap member 2. The body member 1 comprises a cylindrical projection 10, and an expanded holder portion 11 formed integral with one end of the cylindrical projection 10. The cylindrical projection 10 has a smooth outer surface and is terminated at an outwardly curved arched portion 12. The cap member 2 is fixedly fastened to the expanded holder portion 11 of the body member 1 by an ultrasonic sealing apparatus (or the application of an adhesive, screw means), having a filling hole 21. When assembled, the cap member 2 forms with the expanded holder portion 11 the aforesaid disk-like head 20.

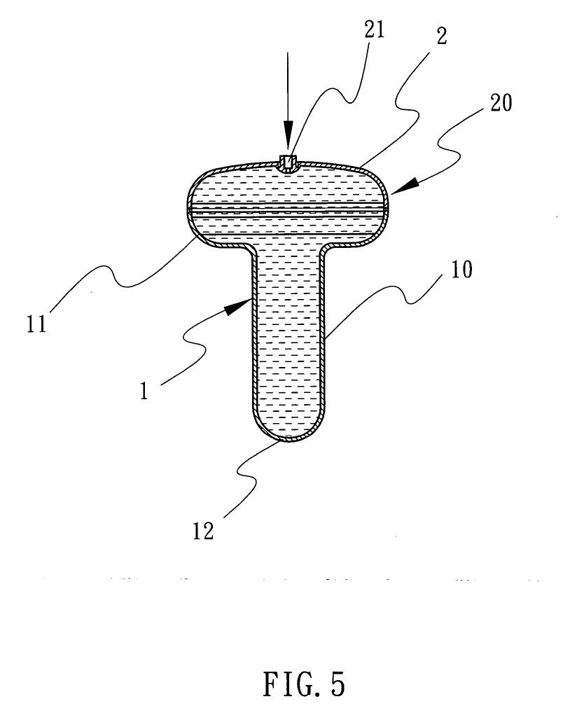

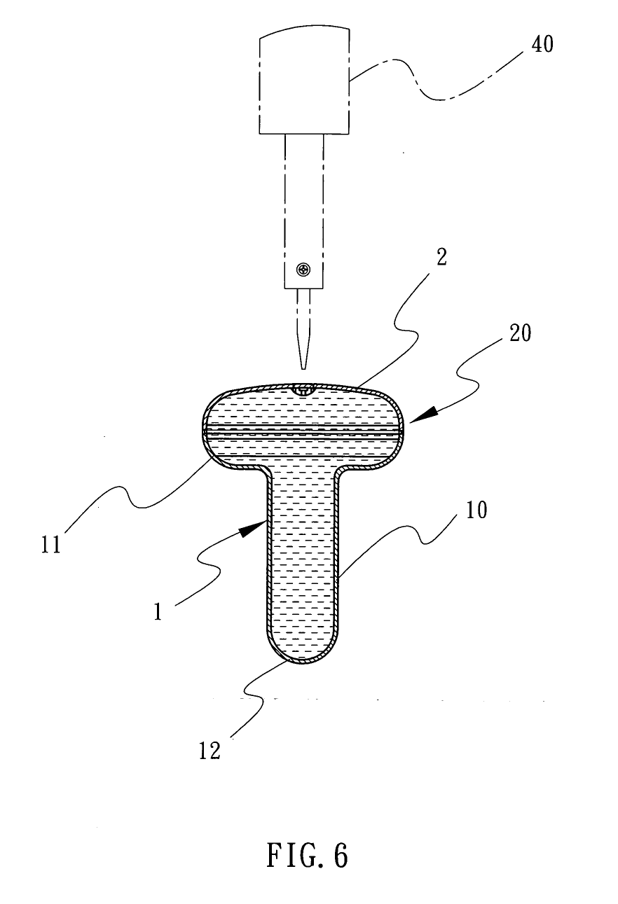

Referring to FIGS. 5 and 6 and FIGS. 3 and 4 again, after connection of the cap member 2 to the body member 1, a low freezing point fluid 30 is filled through the filling hole 21 into the inside of the body member 1, and then a heat sealing apparatus for example, an electric iron is used to seal the filling hole 21.

As indicated above, the invention provides an anus insert, which is a hollow member filled with a low freezing point fluid, having a cylindrical projection 10 for insertion into the anus, and a disk-like head 20 at one end of the cylindrical projection 10 for the holding of the user's hand to insert the cylindrical projection 10 into the anus. After insertion of the anus insert into the anus, the low freezing point fluid is caused to become unfrozen gradually. Therefore, the use of the anus insert to treat hemorrhoids does not cause anus tissues to decay. This device is convenient in use, and washable for repeated use. It is helpful to relieve pain, and causes creation of dense, firm scar tissue in response to previous tissue disease.

The aforesaid low freezing point fluid 30 can be selected from glycerin, water, or anti-freezing agent.

The disk-like head 20 also has a smooth outer surface. After insertion of the anus insert, the disk-like head 20 is kept in close contact with the external hemorrhoids. Therefore, mixed hemorrhoids can be well treated.

Further, the diameter and length of the cylindrical projection 10 must fit the structure of the anus. The disk-like head 20 may be may be in any of a variety of shapes or sizes, suitable for keeping in close contact with the external part of hemorrhoids.

A prototype of anus insert has been constructed with the features of FIGS. 1˜6. The anus insert functions smoothly to provide all of the features discussed earlier.

Although particular embodiments of the invention have been described in detail for purposes of illustration, various modifications and enhancements may be made without departing from the spirit and scope of the invention. Accordingly, the invention is not to be limited except as by the appended claims.

What is claimed is:

1. An anus insert comprising a cylindrical projection, a disk-like head disposed at one end of said cylindrical projection, and a low freezing point fluid filled in an enclosed space defined inside said disk-like head and said cylindrical projection.

2. The anus insert as claimed in claim 1, wherein said disk-like head is formed integral with one end of said cylindrical projection.

3. The anus insert as claimed in claim 1, which is formed of two separate members that are fastened together by means of one of a group of methods including ultrasonic sealing, the application of an adhesive, screw means.

4. The anus insert as claimed in claim 1, wherein said head comprises an expanded holder portion formed integral with one end of said cylindrical front projection, and a cap member fixedly fastened to said expanded rear holder portion by a ultrasonic sealing apparatus, said cover member having a filling hole sealable with a sealing apparatus.

5. The anus insert as claimed in claim 1, wherein said low freezing point fluid is glycerin.

6. The anus insert as claimed in claim 1, wherein said head has a smooth outer surface.
